(19) United States
(12) Patent Application Publication
(10) Pub. No.: US 2023/0256003 A1
(43) Pub. Date: Aug. 17, 2023

(54) PHARMACEUTICAL COMPOSITION FOR PREVENTING OR TREATING SEPSIS CONTAINING FUNCTIONALIZED TRANSITION METAL DICHALCOGENIDE

(71) Applicant: INDUSTRY-UNIVERSITY COOPERATION FOUNDATION HANYANG UNIVERSITY ERICA CAMPUS, Gyeonggi-do (KR)

(72) Inventors: Jong-Ho Kim, Gyeonggi-do (KR); Chul-Su Yang, Gyeonggi-do (KR); DaBin Yim, Gyeonggi-do (KR)

(21) Appl. No.: 18/009,665

(22) PCT Filed: Jun. 24, 2021
(86) PCT No.: PCT/KR2021/007973

§ 371 Date: Dec. 9, 2022

(30) Foreign Application Priority Data
Jun. 25, 2020 (KR) 10-2020-0077609
Jun. 23, 2021 (KR) 10-2021-0081531

(51) Int. Cl. A61K 31/765; A61K 9/70; A61P 31/00; A61K 47/02; A61P 39/06; A61P 29/00

(52) U.S. Cl. CPC A61K 31/765; A61K 9/70; A61P 31/00; A61K 47/02; A61P 39/06; A61P 29/00

(57) ABSTRACT

A 2D TMD nanosheet functionalized with an amphiphilic block polymer compound of the present disclosure has scavenging activity for intracellular and mitochondrial ROS and the scavenging activity is maintained well at low pH, and further, inhibits the excessively increased secretion of inflammatory cytokines in microbial infection, exhibits antibacterial activity, and can increase survivability and prevent aggravation of symptoms in an animal model of sepsis, it can be provided as an anti-inflammatory/antioxidant agent for prevention and treatment of sepsis and septic shock.

### TECHNICAL FIELD

The present disclosure relates to an anti-inflammatory use and a prophylactic or therapeutic use for sepsis and septic shock of a functionalized transition metal dichalcogenide (TMD).

### BACKGROUND ART

Sepsis is caused by the abnormal defensive action of the body against microbial infection. It is associated with the activation of macrophages and the excessive production of inflammation-related factors, reactive oxygen species (ROS) and reactive nitrogen species (RNS) resulting therefrom, which result in severe inflammatory responses throughout the body. Systemic inflammatory response syndrome (SIRS) is diagnosed when two or more symptoms of body temperature above 38° C., body temperature below 36° C., respiratory rate exceeding 24 times per minute (tachypnea), heart rate exceeding 90 beats per minute (tachycardia) and increased or significantly decreased number of white blood cells occur, and sepsis is diagnosed when the systemic inflammatory response syndrome is one caused by microbial infection. Sepsis may result in septic shock. Severe sepsis causes poor function of organs (heart, kidneys, liver, brain, lungs, etc.) and even shock when aggravated. Sepsis may be caused by various pathogens. Although it is usually caused by bacteria, it may also be caused by viruses or fungi. Also, sepsis is often caused by postoperative Infection. The risk of death from sepsis caused by infection or postoperative infection is 40-90%.

It is understood that sepsis results from the complicated interactions between the invading pathogens, the immune system of the host, inflammations and the coagulation system. Both the degree of response of the host and the characteristics of the pathogens has important effects on the prognosis of sepsis. Organ failure observed in sepsis is caused by the inappropriate response of the host to the pathogens. Excessive response of the host to the pathogens may cause damage to the organs of the host. Based on this concept, antagonists against the pro-inflammatory cytokines TNF-α, IL-1β, IL-6, etc., which play a major role in the inflammatory response of the host, have been attempted as therapeutic agents for sepsis, but most of them were unsuccessful. In addition, development of effective antioxidant therapy for sepsis has been attempted to scavenge ROS and RNS, but no effective therapeutic agent has been developed yet. Therefore, there is a need of a new therapeutic agent for treating sepsis and septic shock.

Meanwhile, transition metal dichalcogenides (TMDs) are naturally abundant compounds with the formula MX₂ (M: transition metal, X: chalcogen element) wherein unit layers are stacked. TMDs have a layered structure liked the well-known graphite, and can be obtained as 2D single layers like graphene because the bonding between the layers is weak.

The TMD compounds having a 2D layered structure are also highly applicable as semiconductor logic devices and electrochemical catalysts, including the applicability shown in researches about graphene. Recently, the applicability of 2D TMDs functionalized with amphiphilic block polymer compounds for antioxidant use has been proposed.

### DISCLOSURE OF THE INVENTION

### Technical Problem

The present disclosure is directed to providing an anti-inflammatory pharmaceutical composition of a 2D TMD functionalized with an amphiphilic polymer compound, and a pharmaceutical composition for preventing or treating sepsis and septic shock, which contains the TMD.

However, the problem to be solved by the present disclosure is not limited to that mentioned above. Other problems not mentioned above will be clearly understood by those having ordinary knowledge in the art from the following description.

### Technical Solution

The present disclosure provides a pharmaceutical composition for preventing or treating sepsis or septic shock, which contains a 2D transition metal dichalcogenide (TMD) functionalized with an amphiphilic block polymer compound including a hydrophilic block and a hydrophobic block as an active ingredient.

The present disclosure also provides an anti-inflammatory pharmaceutical composition, which contains the 2D transition metal dichalcogenide (TMD) functionalized with the amphiphilic block polymer as an active ingredient.

In an exemplary embodiment of the present disclosure, the hydrophilic block refers to a moiety of a specific polymer compound which has strong affinity for water and thus is dissolved in water. It may be polyethylene oxide (PEO) or polyethylene glycol (PEG).

In another exemplary embodiment of the present disclosure, the hydrophobic block refers to a moiety of a specific polymer compound which lacks affinity for water and thus is not dissolved in water. It may be poly(ε-caprolactone) (PCL).

In another exemplary embodiment of the present disclosure, the amphiphilic block polymer compound refers to a polymer compound or a copolymer including both a hydrophilic moiety (block) and a hydrophobic moiety (block) in the polymer compound or copolymer. It may be a block polymer compound or a block copolymer including PEG as a hydrophilic block and PCL as a hydrophobic block (“PCL-b-PEG”), and may have a structure of Chemical Formula 1:

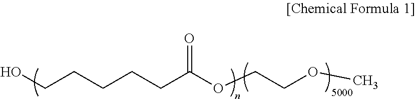

In another exemplary embodiment of the present disclosure, n in Chemical Formula 1 may be an integer from 400 to 3000, specifically an integer from 460 to 2000.

In another exemplary embodiment of the present disclosure, the TMD may be one or more selected from a group consisting of molybdenum disulfide (MoS₂), tungsten disulfide (WS₂), molybdenum diselenide (MoSe₂) and tungsten diselenide (WSe₂), specifically tungsten disulfide, molybdenum diselenide or tungsten diselenide, more specifically tungsten disulfide.

In another exemplary embodiment of the present disclosure, the functionalized 2D TMD may have a thickness of 1-50 nm, specifically 1-10 nm, more specifically 3-5 nm.

In another exemplary embodiment of the present disclosure, the functionalized 2D TMD may have a lateral length of 1-500 nm, specifically 1-100 nm, more specifically 30-43 nm.

In another exemplary embodiment of the present disclosure, the functionalized 2D TMD may have the activity of scavenging intracellular and/or mitochondrial ROS and/or RNS.

In another exemplary embodiment of the present disclosure, the functionalized 2D TMD may inhibit the secretion of an inflammatory cytokine. The inflammatory cytokine may be TNF-α, IL-1β, IL-6, IL-18 or IL-12p40, and the functionalized 2D TMD may not affect the secretion of an anti-inflammatory cytokine.

The present disclosure also provides a method for preventing or treating sepsis or septic shock, which includes a step of administering the functionalized 2D TMD to a subject, wherein the subject may be any mammal exhibiting acute inflammatory response to microbial infection, without limitation, specifically human.

In an exemplary embodiment of the present disclosure, the administration may be intravenous administration or intraperitoneal administration.

### Advantageous Effects

A 2D TMD nanosheet of the present disclosure functionalized with an amphiphilic block polymer compound has superior activity of scavenging intracellular ROS and RNS and mitochondrial ROS, and the scavenging activity is maintained consistently at low pH. In addition, it can increase survivability in an animal model of sepsis and prevent the aggravation of symptoms by inhibiting the excessively increased secretion of inflammatory cytokines due to microbial infection and reducing the number of pathogens. Therefore, it can be provided as an anti-inflammatory/antioxidant agent for preventing and treating sepsis and septic shock.

### BRIEF DESCRIPTION OF THE DRAWINGS

FIG. 1 shows the structure of a biocompatible TMD nanosheet. a) is a schematic representation of application of an exfoliated TMD nanosheet functionalized with PCL-b-PEG for treatment of sepsis, b)-d) show the TEM images of TMD nanosheets, e)-g) show the AFM images of TMD nanosheets, and h) shows the FT-IR spectra of TMD nanosheets functionalized with PCL-b-PEG.

FIG. 2 shows a result of investigating the ROS and RNS scavenging activity of TMD nanosheets. a) shows a result of investigating the sustainability of radical scavenging activity of TMD nanosheets and Trolox, b) shows a result of investigating the radical scavenging activity of TMD nanosheets at different pHs, c) shows the hydrogen peroxide scavenging activity of TMD nanosheets depending on the fluorescence intensity of TAOH, d) shows the hydroxyl radical scavenging activity of TMD nanosheets depending on the fluorescence intensity of TAOH, e) shows the superoxide scavenging activity of TMD nanosheets depending on the absorbance of WST-8 formazan, f) shows the nitric oxide scavenging activity of nanosheets depending on the absorbance of Griess reagent, and g) shows the equilibrium constants of TMD nanosheets for scavenging ROS and RNS.

FIG. 3 shows a result of investigating the scavenging activity of TMD nanosheets for mitochondrial and intracellular ROS and RNS. a) shows a result of investigating the cytotoxicity of TMD nanosheets in BMDMs, b) shows a result of investigating the mitochondrial ROS scavenging activity of TMD nanosheets in LPS-induced inflammatory BMDMs, c) shows a result of investigating the intracellular hydrogen peroxide scavenging activity, d) shows a result of investigating the intracellular hydroxyl radical scavenging activity, e) shows a result of investigating the intracellular superoxide scavenging activity, and f) shows a result of investigating the intracellular nitric oxide scavenging activity. All experiments were repeated 3 times independently, and all the data are represented as mean±SD (* p<0.05; ** p<0.01; *** p<0.001).

FIG. 4 shows a result of investigating the effect of TMD nanosheets on the expression level of ROS-producing enzymes and proteins in BMDMs. a)-c) show a result of investigating the activity of NADPH oxidase, d)-f) show the activity of complex I, g)-i) show the activity of complex III, j) shows the IB analysis result using αgp91phox, αp22phox and αp47phox (NOX subunit), and k) shows the IB analysis result using αNDUFA9 and αNDUFB8 (subunits of complex I), αUQCRC2 and αUQCRQ (subunits of complex III) and α-actin. All experiments were repeated 3 times independently, and all the data are represented as mean±SD.

FIG. 5 shows a result of investigating the effect of TMD nanosheets on the secretion of inflammatory cytokines. a)-f) show a result of investigating the effect on acute inflammatory cytokines (TNF-α and IL-1β), and g)-1) show a result of investigating the secretion level of chronic inflammatory cytokine (IL-6 and IL-12p40) by treating LPS-induced inflammatory BMDMs with TMD nanosheets and analyzing the supernatant by ELISA. All experiments were repeated 3 times independently, and all the data are represented as mean±SD (* p<0.05; ** p<0.01; *** p<0.001).

FIG. 6 shows a result of investigating the ROS scavenging activity of a WS₂ nanosheet in human monocytes infected with bacteria for 18 hours as well as inflammatory response and antibacterial effect. a)-c) show a result of investigating the mitochondrial ROS scavenging activity, d)-f) show a result of investigating the intracellular superoxide scavenging activity, g)-i) show a result of investigating the intracellular hydrogen peroxide scavenging activity of a WS₂ nanosheet in human monocytes infected with E. coli (MOI=50), S. aureus (MOI=20) or P. aeruginosa (MOI=20), j)-1) show a result of investigating the intracellular nitric oxide scavenging activity of a WS₂ nanosheet, m)-o) show a result of investigating the effect on the acute inflammatory cytokine TNF-α, p)-r) show a result of investigating the effect on the chronic inflammatory cytokine IL-6, and s)-u) show a result of investigating the secretion level of chronic IL-12p40 after pretreating human monocytes with a WS₂ nanosheet for 45 minutes. In addition, v) shows the survival rate of P. aeruginosa and w) shows the survival rate of S. aureus after treating human monocytes infected with the bacteria with a WS₂ nanosheet. All experiments were repeated 3 times independently, and all the data are represented as mean±SD (* p<0.05; ** p<0.01; *** p<0.001).

FIG. 7 shows a result of investigating the therapeutic effect of a WS2 nanosheet for CLP-induced bacteremic mice. a) show a result of intraperitoneally injecting a WS2 nanosheet at 1 hour and 11 hours and then comparing the survival rate of septic mice with that of CLP+PBS mice (n=15 mice per group), b) shows a result of investigating the level of inflammatory cytokines (TNF-α, IL-1β, IL-6 and IL-12p40) in the serum, c) shows a result of investigating the number of bacterial colonies in the blood (top) and peritoneal fluid (bottom) of septic mice 24 hours after treatment, d) shows the typical H&E staining images of the lung, liver and spleen of mice, and e) shows the histological scores (ranging from 0 to 4) corresponding to the degree of inflammation in the H&E staining images. All experiments were repeated 3 times independently, and all the data are represented as mean±SD (* p<0.05; ** p<0.01; *** p<0.001).

FIG. 8 shows a result of investigating the in-vivo pharmacokinetics of a WS2 nanosheet. After intravenously injecting a WS2 nanosheet including IR783 (8.5 nM) to a mouse, the fluorescence images of the kidney, lung, liver and spleen were obtained using an IVIS spectrum-chromatography (CT) system.

FIG. 9 shows the images of exfoliated TMD nanosheets in solutions (a) and a result of investigating the zeta potential (*-potential) of WS₂, MoSe₂ and WSe₂ nanosheets.

FIG. 10 shows the FT-IR spectrum of PCL-b-PEG.

FIG. 11 shows the XPS spectra of exfoliated TMD nanosheets functionalized with PCL-b-PEG in aqueous solutions (a: W 4f of WS₂ nanosheet, b: Mo 3d of MoSe₂ nanosheet, c: W 4f of WSe₂ nanosheet).

FIG. 12 a) shows the UV-Vis absorption spectra of exfoliated TMD nanosheets functionalized with PCL-b-PEG, b) shows the absorption spectra of exfoliated TMD nanosheets in aqueous solutions depending on time, c) shows a result of measuring the ratio of the absorbance of exfoliated TMD nanosheets left alone for 14 days and that immediately after exfoliation at excitation wavelengths, and d) shows the Raman spectra of exfoliated WS₂, MoSe₂ and WSe₂ nanosheets functionalized with PCL-b-PEG (wavelength: 532 nm).

FIG. 13 shows a result of investigating the sustainability of the hydroxyl radical scavenging activity of exfoliated WS₂, MoSe₂ and WSe₂ nanosheets functionalized with PCL-b-PEG.

FIG. 14 shows a result of investigating the sustainability of the nitric oxide scavenging activity of exfoliated WS₂, MoSe₂ and WSe₂ nanosheets functionalized with PCL-b-PEG.

FIG. 15 shows that PCL-b-PEG itself lacks ROS and RNS scavenging activity (a) hydrogen peroxide, b) hydroxyl radical, c) superoxide, d) nitric oxide).

FIG. 16 shows the hydrogen peroxide scavenging activity of a) WS₂, b) MoSe₂ and c) WSe₂ nanosheets at various concentrations and d) the concentration-dependent scavenging activity of the TMD nanosheets.

FIG. 17 shows the hydroxyl radical scavenging activity of a) WS₂, b) MoSe₂ and c) WSe₂ nanosheets at various concentrations and d) the concentration-dependent scavenging activity of the TMD nano sheets.

FIG. 18 shows the superoxide scavenging activity of a) WS₂, b) MoSe₂ and c) WSe₂ nanosheets at various concentrations and d) the concentration-dependent scavenging activity of the TMD nano sheets.

FIG. 19 shows the nitric oxide scavenging activity of a) WS₂, b) MoSe₂ and c) WSe₂ nanosheets at various concentrations and d) the concentration-dependent scavenging activity of the TMD nano sheets.

FIG. 20 shows the Langmuir adsorption isotherms for identifying the equilibrium constants (Kₛ) of WS₂, MoSe₂ and WSe₂ nanosheets at various concentrations for scavenging a) hydrogen peroxide, b) hydroxyl radical, c) superoxide and d) nitric oxide.

FIG. 21 shows the XPS spectra of a WS₂ nanosheet after scavenging of ROS and RNS (a) W 4f spectrum of WS₂ nanosheet immediately after exfoliation, b) W 4f spectrum of WS₂ nanosheet after hydrogen peroxide scavenging, c) W 4f spectrum of WS₂ nanosheet after nitric oxide scavenging, d) N1s spectrum of WS₂ nanosheet after nitric oxide scavenging).

FIG. 22 shows the a) ROS and b) RNS scavenging reactions of 2D TMDs. The abbreviations in the reaction formulas are as follows: M (transition metal atom), X (chalcogen atom), HO-MX₂ (hydroxylated TMD), XV-MX (chalcogen-vacant TMD), Red-ROS (reduced ROS), O-MX (oxidized TMD), ON-MX (nitrosylated TMD), N-MX (N-bearing TMD).

FIG. 23 shows a result of measuring the ROS and RNS scavenging activity of TMD nanosheets in LPS-induced inflammatory BMDMs by FACS. a)-c) show a result of investigating the mitochondrial ROS scavenging activity, d)-f) show a result of investigating the intracellular superoxide scavenging activity, g)-i) show a result of investigating the intracellular hydrogen peroxide scavenging activity, and j)-1) show a result of investigating the intracellular nitric oxide scavenging activity. The BMDMs were stimulated with LPS for 30 minutes to induce inflammation and then treated with TMDs for 45 minutes.

FIG. 24 shows a result of investigating the effect of TMD nanosheets on the expression of an acute inflammatory cytokine (IL-18) by treating BMDMs, wherein inflammation has been induced by stimulating with LPS/ATP for 1 hour, with TMD nanosheets and analyzing the supernatant by ELISA (a: WS₂, b: MoSe₂, c: WSe₂). All experiments were repeated 3 times, and all the data are represented as mean±SD. The significant difference from LPS/ATP+SC is shown (* p<0.05; ** p<0.01; *** p<0.001).

FIG. 25 shows a result of investigating the effect of TMD nanosheets on the secretion of an anti-inflammatory cytokine (IL-10) by treating BMDMs, wherein inflammation has been induced by stimulating with LPS for 18 hours, with LPS for 18 hours and analyzing the supernatant by ELISA (a: WS₂, b: MoSe₂, c: WSe₂). Data obtained by repeating experiments 3 times are represented as mean±SD. No significant difference was observed from LPS+SC.

FIG. 26 shows a result of investigating the effect of endocytosis of a TMD nanosheet on the expression of inflammatory cytokines. Raw264.7 cells were cultured in a medium containing cytochalasin D (endocytosis inhibitor, Cyto D, 5, 10 or 20 μM) for 30 minutes and then treated with a WS₂ nanosheet for 45 minutes. Then, after stimulating the Raw264.7 cells with LPS for 18 hours, the supernatant was obtained and analyzed by ELISA. Data obtained by repeating experiments 3 times are represented as mean±SD and the significant difference from LPS+SC is shown (* p<0.05; ** p<0.01; *** p<0.001).

FIG. 27 shows a result of investigating the ROS scavenging activity and inflammatory cytokine inhibition activity of a WS₂ nanosheet in human monocytes. a) shows a result of measuring the mitochondrial ROS scavenging activity of a WS₂ nanosheet at various concentrations by FACS, b) shows the intracellular superoxide scavenging activity, and c) shows a result of measuring the intracellular hydrogen peroxide scavenging activity. The human monocytes were stimulated with LPS for 30 minutes. In addition, d)-e) show a result of investigating the level of cytokines by analyzing the supernatant of human monocytes which have been treated with the WS₂ nanosheet for 45 minutes and stimulated with LPS for 18 hours by ELISA (d: TNF-α, e: IL-6 and IL-12p40). Data obtained by repeating experiments 3 times are represented as mean±SD and the significant difference from LPS+SC is shown (* p<0.05; ** p<0.01; *** p<0.001).

FIG. 28 shows a result of investigating the therapeutic effect of a WS₂ nanosheet in a CLP-induced bacteremic mouse model. a) shows the survival rate of the mice (n=10) to which a WS₂ sheet has been administered intravenously (i.v.) prior to treatment with CLP, and b) shows the survival rate of the mice (n=10) to which the sheet has been administered intravenously (i.v.) 1 hour after the treatment with CLP.

FIG. 29 shows a result of investigating the therapeutic effect of a WS₂ nanosheet in a CLP-induced bacteremic mouse model. a) shows the survival rate of the mice (n=10) to which a WS₂ sheet has been administered intraperitoneally (i.p.) prior to treatment with CLP, b) shows the survival rate of the mice (n=10) to which the WS₂ nanosheet has been administered intravenously (i.v.) 1 hour after the treatment with CLP, and c) shows the survival rate of the mice (n=10) to which the WS₂ nanosheet has been administered intravenously (i.v.) for 1 hour before and after the treatment with CLP. Statistical difference from the CLP+PBS mice to which PBS has been administered is shown (log-rank test).

FIG. 30 shows a result of investigating the therapeutic effect of a WS₂ nanosheet in a CLP-induced polymicrobial septic mouse model. a) shows the survival rate of the mice (n=10) to which the WS₂ nanosheet has been administered intraperitoneally (i.p.) 1 hour and 11 hours after infection with E. coli, b) shows a result of obtaining blood and peritoneal fluid 24 hours and 2 days after the infection and counting the number of E. coli colonies, c) shows the survival rate of the mice (n=10) to which the WS₂ nanosheet has been administered intraperitoneally (i.p.) 1 hour and 11 hours after infection with S. aureus, d) shows a result of obtaining blood and peritoneal fluid 24 hours and 2 days after the infection and counting the number of S. aureus colonies, e) shows the survival rate of the mice (n=10) to which the WS₂ nanosheet has been administered intraperitoneally (i.p.) 1 hour and 11 hours after infection with P. aeruginosa, and f) shows a result of obtaining blood and peritoneal fluid 24 hours and 2 days after the infection and counting the number of P. aeruginosa colonies. Significant difference from the mice infected with the bacteria without any treatment is shown (* p<0.05; ** p<0.01; *** p<0.001).

FIG. 31 shows the UV-Vis absorption and fluorescence spectra of IR783.

FIG. 32 shows the UV-Vis absorption spectrum (a) and fluorescence spectrum (b) of WS₂/IR783.

WS₂/IR783.

FIG. 33 shows a result of investigating the fluorescence stability of WS₂/IR783 with time.

FIG. 34 shows a result of investigating the pharmacokinetics of an intraperitoneally administered WS₂ nanosheet. a) shows fluorescence the images of the kidney, lung, liver and spleen taken out from a mouse to which a WS₂ nanosheet including IR783 (8.5 nM) has been administered intraperitoneally, and b) shows a result of quantifying the fluorescence intensity from each organ using an IVIS spectrum-chromatography (CT) system.

### BEST MODE FOR CARRYING OUT THE INVENTION

The present disclosure was completed based on the finding that a 2D transition metal dichalcogenide (2D TMD) functionalized with an amphiphilic block polymer compound has scavenging activity for reactive oxygen species and reactive nitrogen species and has therapeutic effect for sepsis caused by acute microbial infection.

Specifically, the inventors of the present disclosure have prepared exfoliated 2D TMD (WS₂, MoSe₂ or WSe₂) nanosheets functionalized with PCL-b-PEG in aqueous solutions and found out that they are biocompatible through structural analysis (see Example 1).

Then, the inventors of the present disclosure have confirmed the sustainability of scavenging activity for various radicals by comparing the three TMD nanosheets with Trolox through ABTS radical scavenging activity assay. As a result, the three TMD nanosheets showed consistent ROS and RNS scavenging activity at significantly lower concentrations as compared to Trolox. In addition, the three TMD nanosheets showed superior activity for scavenging of hydrogen peroxide, hydroxyl radical, superoxide and nitric oxide. In particular, the WS₂ nanosheet showed better scavenging activity than other nanosheets and the W-based TMD nanosheets showed better superoxide scavenging activity than the Mo-based TMD nanosheets (see Example 2).

In severe sepsis, abnormal inflammatory response decreases the pH of cells and tissues than normal levels. Accordingly, it is expected from the sustainability of the radical scavenging activity of the TMD nanosheets even under low pH conditions that they can be effective in treating sepsis in vivo. In addition, the effect of the 2D TMD nanosheets was investigated in inflammation-induced cells.

For this, the cytotoxic of the three TMD nanosheets was investigated experimentally and the level of mitochondrial ROS and intracellular hydrogen peroxide, hydroxyl radical, superoxide and nitric oxide was investigated after treating LPS-induced inflammatory BMDMs with the TMD nanosheets. As a result, it was confirmed that the TMD nanosheets have superior scavenging activity for intracellular ROS and RNS, and the WS2 nanosheet has the best activity. The three TMD nanosheets scavenged ROS and RNS, but were not involved in the ROS generation mechanism in the mitochondria (see Example 3).

In addition to the ROS and RNS scavenging activity in inflammation-induced cells, it was confirmed that the TMD nanosheets can decrease the secretion of acute and chronic inflammatory cytokines in a concentration-dependent manner and have no effect on the secretion of anti-inflammatory cytokines. In addition, they also scavenged intracellular and mitochondrial ROS and inhibited the expression of pro-inflammatory cytokines in human monocytes infected with E. coli, P. aeruginosa or S. aureus (see Example 4).

Also, the inventors of the present disclosure have found out that the treatment of human monocytes infected with E. coli, P. aeruginosa or S. aureus with the WS₂ nanosheet reduces the number of bacteria in the monocytes. That is to say, it was confirmed that the TMD nanosheets can be used as effective therapeutic agents for sepsis because they can consistently scavenge ROS and RNS, decrease the secretion of inflammatory cytokines and reduce the number of bacteria in infected cells (see Example 4).

The therapeutic effect of the TMD nanosheets for sepsis was investigated again in a CLP-induced bacteremic mouse model. Specifically, it was confirmed that the number of bacteria is decreased remarkably and survival rate is increased when the TMD nanosheets are injected intravenously or intraperitoneally to CLP-induced bacteremic mice. In addition, it was confirmed from the histopathological conditions of kidney, lungs, liver and spleen that the TMD nanosheets can prevent the aggravation of sepsis in the bacteremic mice (see Example 5).

Meanwhile, the TMD nanosheets showed different behaviors depending on administration routes. They were discharged from the body in 3 days when injected intravenously, but could remain in the body for 10 days when they were injected intraperitoneally. Accordingly, the administration route of the TMD nanosheets can be determined in consideration of the conditions and/or pathologies of the subject (see Example 5).

Therefore, the inventors of the present disclosure provide a prophylactic or therapeutic use for an inflammatory disease and a prophylactic or therapeutic use for sepsis and/or septic shock of an exfoliated 2D TMD nanosheet functionalized with an amphiphilic block polymer compound.

In the present specification, the “prevention or treatment of sepsis or septic shock” refers to the reduction, improvement or removal of clinical symptoms associated with sepsis and all or some symptoms of a condition associated with multiple organ dysfunction syndrome (e.g., fever of various grades, hypoxemia, tachycardia, endothelitis, myocardial infarction, delirium, vascular collapse, organ damage, acute respiratory distress syndrome, coagulopathy, heart failure, renal failure, shock and/or lethargy).

In the present specification, the “functionalization” includes changing, improving or modifying the physical, chemical or biological properties (e.g., interfacial characteristics) by introducing, treating, coating or binding a physical means (heat, pressure, vibration, light, etc.) or a chemical means (specific compound, polymer, functional group, etc.). For example, introduction of a specific polymer compound for hydrophobic interaction may be a kind of functionalization.

In the present disclosure, prevention refers to any action of delaying infection by sepsis-inducing bacteria or delaying the onset of inflammatory response and disease caused by the infection by administering a pharmaceutical composition according to the present disclosure, and treatment refers to any action of improving or favorably changing the inflammatory response against the infection or the symptoms of infection or sepsis or septic shock by administering the pharmaceutical composition according to the present disclosure.

In the present disclosure, the pharmaceutical composition may further contain, in addition to the functionalized 2D TMD nanosheet, one or more known antibiotic and/or anti-inflammatory agent, and may further contain a suitable carrier, excipient or diluent commonly used for preparation of a pharmaceutical composition.

In the present disclosure, the “carrier”, which is also called a vehicle, refers to a compound which facilitates the introduction of a protein or a peptide into a cell or a tissue. For example, dimethyl sulfoxide (DMSO) is a carrier commonly used to facilitate the introduction of many organic substances into the cells or tissues of an organism.

In the present disclosure, the “diluent” is defined as a compound which stabilizes the biologically active form of a target protein or peptide and is diluted in water wherein the protein or peptide is dissolved. A salt dissolved in a buffer solution is used as a diluent. Phosphate-buffered saline, which mimics the salt form of human body fluid, is a commonly used buffer solution. A buffer diluent rarely alters the biological activity of a compound because a buffer salt can control the solution pH at low concentration. The compound used in the present disclosure can be administered to a human patient either alone or in combination with another ingredient in combination therapy or can be administered as being mixed with a suitable carrier or excipient in a pharmaceutical composition.

In addition, the pharmaceutical composition according to the present disclosure may be formulated into a powder, a granule, a tablet, a capsule, a suspension, an emulsion, a syrup, an aerosol, a sterile injection solution, etc. according to common methods and, depending on purposes, the composition of the present disclosure may be administered orally or parenterally (e.g., intravenously, subcutaneously, intraperitoneally or topically), specifically parenterally, more specifically intravenously. The administration dosage may be determined adequately by those skilled in the art although it may vary depending on the physical condition and body weight of a patient, the severity of a disease, drug type, administration route and administration period. For example, about 0.001-1000 mg may be administered as a mixture with a pharmaceutically acceptable carrier. The pharmaceutical composition of the present disclosure may be administered once or several times a day as necessary, and may be administered alone or in combination with surgery, hormone therapy, drug therapy or a biological response modulator.

The functionalized 2D TMD nanosheet of the present disclosure may be provided as a quasi-drug composition for the purpose of preventing or improving a microbial infection disease inducing sepsis. The quasi-drug composition of the present disclosure may be used together with another quasi-drug or quasi-drug ingredient according to a common method. The quasi-drug composition may be used in an antibacterial cleanser, a shower foam, a gargle, a wet tissue, a laundry soap, a hand sanitizer, a humidifier filler, a mask, an ointment, a filter filler, etc., although not being limited thereto.

The present disclosure may be changed variously and may have various exemplary embodiments. Hereinafter, specific exemplary embodiments will be described in detail referring to drawings. However, it is not intended to limit the present disclosure to those specific exemplary embodiments, and it should be understood that all modifications, equivalents and substitutes thereof are included in the scope of the present disclosure. When describing the present disclosure, detailed description of the known art will be omitted if it is determined that it may unnecessarily obscure the subject matter of the present disclosure

[Experimental Method]

1. Preparation of TMD Nanosheets

Bulk TMDs (0.6 g of WS2, 0.61 g of MoSe2 and 0.83 g of WSe2) were added to 20 mL of a PCL460-b-PEG5000 solution (2 mg/mL). Then, the mixture was sonicated for 1 hour (pulse-on for 6 sec, pulse-off for 2 sec). The solution was centrifuged at 700 g for 1 hour. After centrifuging the obtained supernatant at 14,500 g for 1 hour, the produced precipitate was centrifuged again after adding water and then washed after centrifuging again. The precipitate was dispersed in 8.5 mL of water and the solution was centrifuged at 2,000 g for 30 minutes. TMD nanosheets were obtained from the supernatant.

2. Measurement of ROS and RNS Scavenging Activity of TMD Nanosheets

2-1. Measurement of H₂O₂ Scavenging Activity

After reacting 150 μL of H₂O₂ (3 mM) and 150 μL of the TMD nanosheet (54 nM) for 8 hours, the TMD nanosheet was removed by centrifugation. Then, after mixing with 100 μL of a FeSO₄ (120 μM) solution and leaving alone for 1 hour, 50 μL of terephthalic acid (TA, 22.5 mM in 0.1 M NaOH) was added and fluorescence emission was measured at 45 minutes with a spectrofluorometer.

2-2. Measurement of Hydroxyl Radical Scavenging Activity

125 μL of H₂O₂ (6 mM) and 125 μL of FeSO₄ (60 μM) were mixed for 30 minutes to generate hydroxyl radicals. After adding 200 μL of the TMD nanosheet (60 nM) to the hydroxyl radical solution, the mixture was stirred at 25° C. for 90 minutes. After adding to TA (50 μL, 30 mM, 0.1 M NaOH), the mixture was agitated for 45 minutes. Then, after removing the TMD nanosheet by centrifugation, fluorescence emission was measured with a spectrofluorometer.

2-3. Measurement of Superoxide Scavenging Activity

Superoxide was generated by reacting xanthine (XAN) with xanthine oxidase (XOD). After mixing 30 μL of XAN (1 mM), 125 μL of the TMD nanosheet (54 nM) and 15 μL of WST-8 (5-(2,4-disulfophenyl)-3-(2-methoxy-4-nitrophenyl)-2-(4-nitrophenyl)-2H-tetrazolium), 30 μL of XOD (0.05 U mL⁻¹) was added. After stirring the mixture at 25° C. for 2 hours and removing the TMD nanosheet through centrifugation, the absorbance of the supernatant was measured using a microplate reader and a UV-Vis spectrometer.

2-4. Measurement of Nitric Oxide Scavenging Activity

NO (nitric oxide) was generated using sodium nitroprusside (SNP). The amount of NO after scavenging by the TMD was measured using an analysis kit (Abcam). Specifically, 150 μL of SNP (20 mM) and 150 μL of the TMD nanosheet (54 nM) were mixed with a 0.1 M PBS solution (pH 7.4) and then reacted by shaking for 2 hours. 7.5 μL of nitrate reductase and 7.5 μL of enzyme cofactor were added to the mixture and reaction was conducted for 1 hour. Then, after conducting reaction with nitrite for 20 minutes by adding 50 μL of Griess reagents R1 and R2, the TMD nanosheet was removed. Then, the absorbance of the supernatant was measured with a microplate reader and a UV-Vis spectrometer.

3. Flow Cytometry of ROS and RNS Production

Cells cultured in SFM (serum-free medium) supplemented with a redox-sensitive dye (2 μM dihydroethidium (DHE for 02 Calbiochem), 10 μM 2′,7′-dichlorofluorescin diacetate (DCFH-DA for H₂O₂; Calbiochem), 10 μM 4,5-diaminofluorescein diacetate (DAF-2DA for NO; Calbiochem) or 5 μM MitoSOX™ Red mitochondrial superoxide indicator (for mitochondrial ROS; Molecular Probes)) were subjected to flow cytometry for measurement of intracellular ROS. The cells were washed quickly with pulse spin and then analyzed immediately using FACSCalibur (BD Biosciences, San Jose, Calif.). Data were quantified using the CellQuest software (BD Biosciences).

4. Measurement of NADPH Oxidase Activity

The production of peroxide in cells was measured by the lucigenin (bis-N-methylacridinium nitrate)-ECL method. Specifically, a cell lysate was mixed with 50 mM PBS (pH 7.0), 1 mM EGTA, 150 mM sucrose and a protease inhibitor. After reaction at 37° C. for 30 minutes, a Krebs-HEPES buffer containing lucigenin (5 μM) and NADPH (100 μM) was added. All data were expressed as relative light units per 1×10⁵ cells.

5. Preparation of Animal Models

CLP (cecal ligation and puncture)- or bacteria-induced sepsis models were prepared using 6-week-old C57BL/6 female mice (Samtako Bio, Gyeonggi-do, Korea).

The CLP-induced mouse model was prepared by intravenously injecting pentothal sodium (50 mg/kg) to the mice, exposing the cecum by preparing a small incision at the center of the abdomen, ligating the cecum below the serosa, preparing two openings using a 22-gauge needle and then closing the abdomen. The survival rate of the mice was monitored for 7 days.

The bacterial infection-induced sepsis model was prepared by culturing E. coli (serotype 086: K61 (B7), ATCC 12701), S. aureus (ATCC 6538) or P. aeruginosa (ATCC 10145) at 37° C. in BHI (brain-heart-infusion) BD (broth medium). Mid-log-phase bacteria (absorbance 0.5) were used. The bacterial culture was isolated and stored at −80° C. until use. The effective concentration of LPS was 50 pg/mL or lower. All the procedures were approved by the Hanyang University Biosafety Committee (Protocol 2014-01).

### EXAMPLES

### Example 1. Preparation and Analysis of 2D TMD Nanosheets Functionalized with Amphiphilic Block Polymer Compound

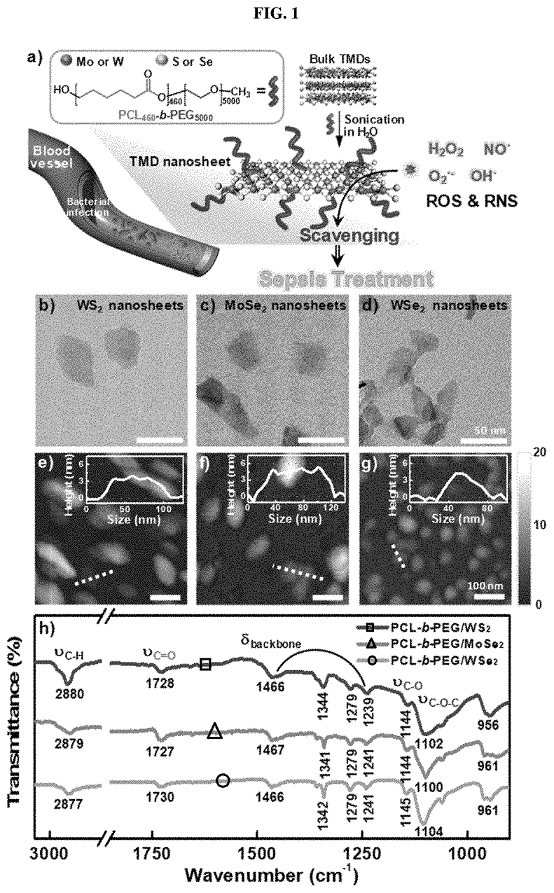

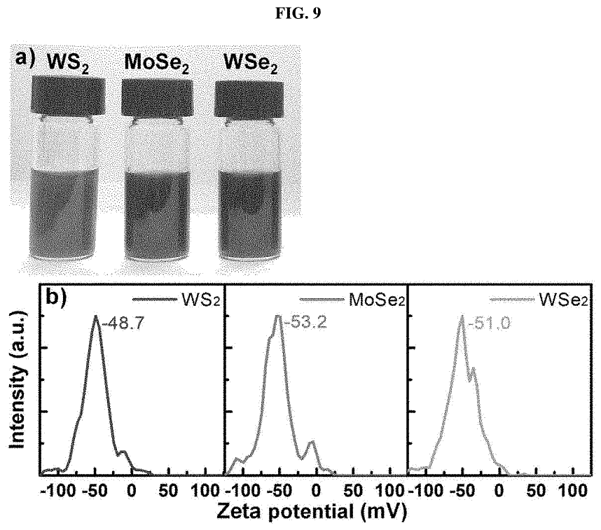

In order to exfoliate ultrathin functionalized 2D TMD nanosheets in aqueous solutions, an amphiphilic poly(ε-caprolactone)-b-poly(ethylene glycol) (PCL-b-PEG) diblock copolymer was used for liquid-phase exfoliation of bulk (3D) WS₂, MoSe₂ or WSe₂ (FIG. 1a). The three TMD nanosheets (WS₂, MoSe₂ and WSe₂) were exfoliated at concentrations of 246, 301 and 394 μg/mL, respectively (FIG. 9a). The negative zeta potential (*-potential) of the exfoliated 2D TMDs is favorable for sustainability of dispersion stability (FIG. 9b).

It was confirmed from the TEM (transmission electron microscopy) and AFM (atomic force microscopy) images of the WS₂, MoSe₂ and WSe₂ nanosheets that each nanosheet has an average lateral length of 37.5 nm and an average thickness of 4 nm (FIG. 1B-g).

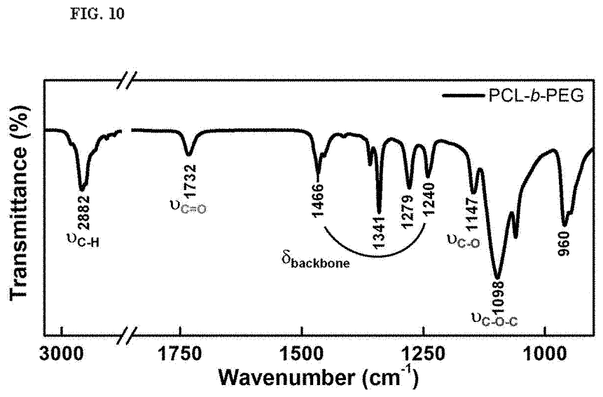

The functionalization of the 2D TMD nanosheet with PCL-b-PEG was identified clearly from FT-IR (Fourier transform infrared) spectra (FIG. 1h and FIG. 10). In addition, the vibrational mode characteristics of C—H, C═O and C—O—C bonds of PCL-b-PEG in the exfoliated WS₂, MoSe₂ and WSe₂ nanosheets were observed at 2882, 1732 and 1098 cm⁻¹, respectively. This result means that the three exfoliated nanosheets have nearly the same size and are functionalized with PCL-b-PEG.

The chemical structure of the 2D TMD nanosheets functionalized with PCL-b-PEG was analyzed by XPS (X-ray photoelectron spectroscopy) (FIG. 11). The WS₂ nanosheet showed the characteristic peaks of the 2H 32.7 and 34.8 eV in the W 4f XPS spectrum. The MoSe₂ nanosheet exhibited distinct peaks of the semiconductor 2H phase at 229.6 and 232.7 eV in the Mo 3d XPS spectrum. The WSe₂ nanosheet showed characteristic peaks at 32.8 and 35.0 eV in the W 4f XPS spectrum, suggesting a hexagonal (2H) structure. W and Mo were oxidized partly during the exfoliation in aqueous solutions.

Unlike the TMD with an octahedral (1T) structure exhibiting cytotoxicity, the TMD nanosheet of the 2H phase has superior biocompatibility in vitro and in vivo. Accordingly, it can be seen that all the three TMD nanosheets functionalized with PCL-b-PEG and exfoliated in aqueous solutions have 2H structures and have superior biocompatibility.

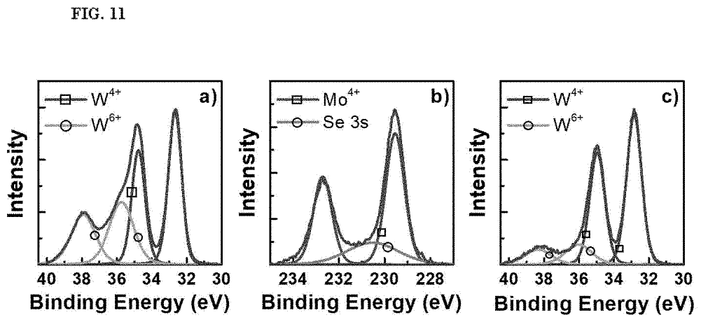

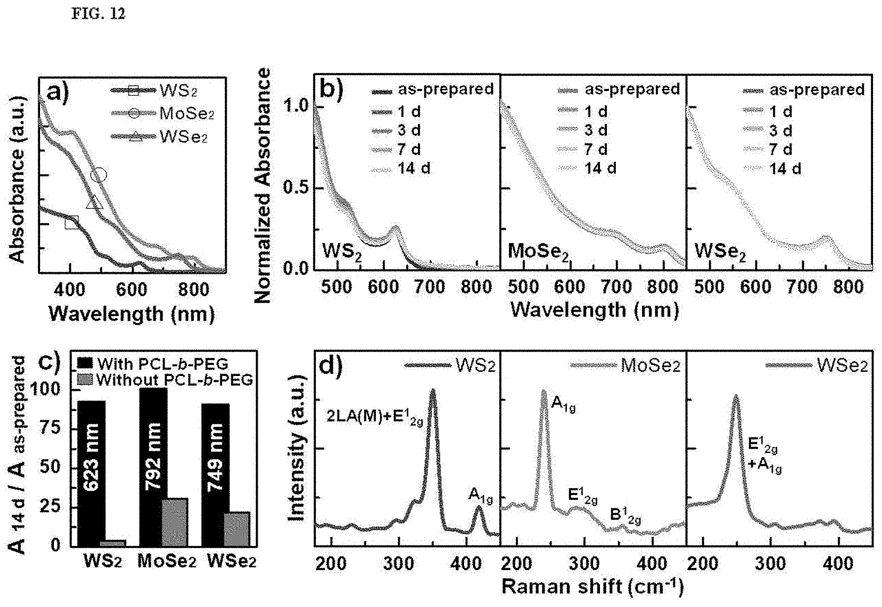

In addition, the 2H structure of the exfoliated TMD nanosheets could be identified from optical properties. The characteristic A excitonic absorption peaks of the WS₂, MoSe₂ and WSe₂ nanosheets were observed at 623, 792 and 749 nm, respectively. Through this, it was clearly confirmed that the exfoliated TMD nanosheets have 2H phases. In addition, the structure of the exfoliated TMD nanosheets was confirmed again by Raman spectroscopy. The intensity ratio of the longitudinal acoustic mode [2LA(M)] peak at 350 cm⁻¹ to the out-of-plane vibrational mode (A_{1g}) peak at 418 cm⁻¹ in the Raman spectrum was larger than 6.7 for the WS₂ nanosheet, indicating that WS₂ was exfoliated very thinly. The peak position in the A_{1g} mode is indicative of the number of MoSe₂ layers. The MoSe₂ nanosheet showed an A_{1g} mode peak at 239 cm⁻¹, which corresponds to MoSe₂ having thin layers. In addition, the WSe₂ nanosheet showed a single peak (248 cm⁻¹) due to degeneration of A_{1g} and E¹_{2g} modes. This means that the WSe₂ nanosheet has been exfoliated well as thin layers (FIG. 12).

### Example 2. Confirmation of ROS and RNS Scavenging Activity of TMD Nanosheets

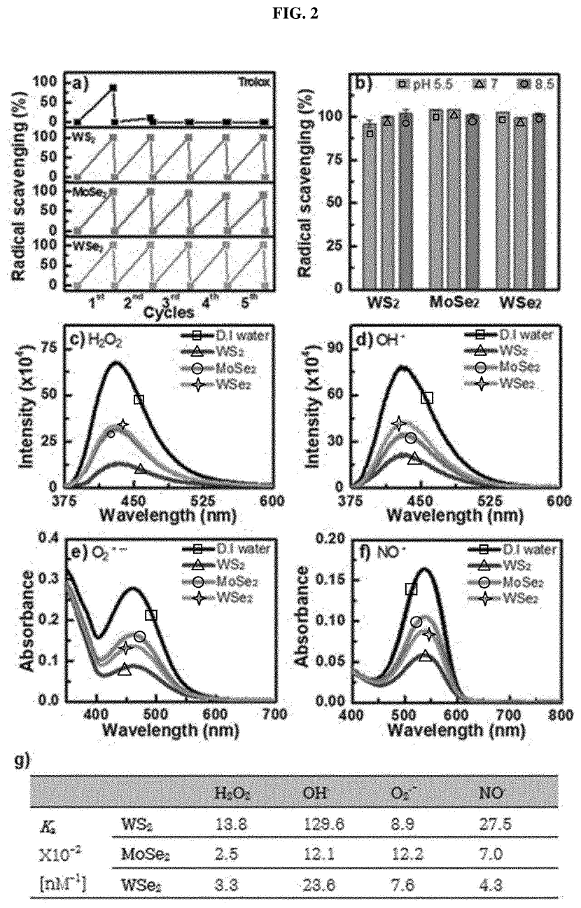

The sustainability of the ROS scavenging activity of the three TMD nanosheets functionalized with PCL-b-PEG was investigated by ABTS (2,2′-azino-bis(3-ethylbenzothiazoline-6-sulfonic acid)) assay. As can be seen from FIG. 2a, whereas 12 μM Trolox showed ROS scavenging activity only for a short time, the WS₂, MoSe₂ and WSe₂ nanosheets showed consistent ROS scavenging activity at a lower concentration (0.012 μM) despite further addition of AB TS radicals.

Then, the ROS scavenging activity of the three TMD nanosheets for hydrogen peroxide was investigated by TA (terephthalic acid) assay. As can be seen from FIG. 2c, the fluorescence intensity of TAOH at 450 nm was decreased significantly in the presence of the WS₂, MoSe₂ or WSe₂ nanosheets, suggesting that H₂O₂ was effectively removed by the nanosheets. In particular, the WS₂ nanosheet showed the best H₂O₂ scavenging activity.

Then, the scavenging activity of the three TMD nanosheets for hydroxyl radicals was investigated by TA assay. As can be seen from FIG. 2d, the fluorescence intensity of TAOH was decreased significantly in the presence of the WS₂, MoSe₂ or WSe₂ nanosheets, suggesting that the hydroxyl radicals produced by Fenton reaction were effectively removed by the TMD nanosheets. In particular, the WS₂ nanosheet removed the hydroxyl radicals the most effectively.

The scavenging activity of the three TMD nanosheets for superoxide was investigated by WST-8 (tetrazolium salt) assay. Superoxide radicals generated by the reaction between xanthine and xanthine oxidase convert WST-8 tetrazolium into WST-8 formazan, and the WST-8 formazan exhibits strong absorbance at 460 nm. As can be seen from FIG. 2e, the absorbance at 460 nm was decreased significantly in the presence of the WS₂, MoSe₂ or WSe₂ nanosheet, indicating that superoxide was effectively removed by the TMD nanosheets. In particular, the W-based TMD nanosheet showed higher scavenging activity for superoxide than the Mo-based TMD.

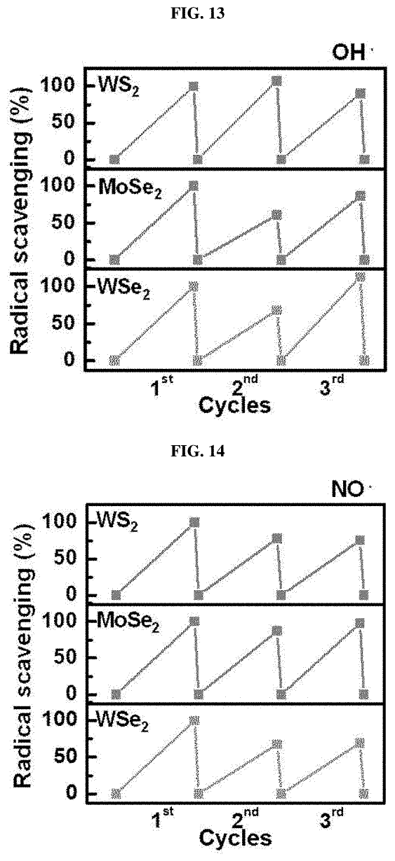

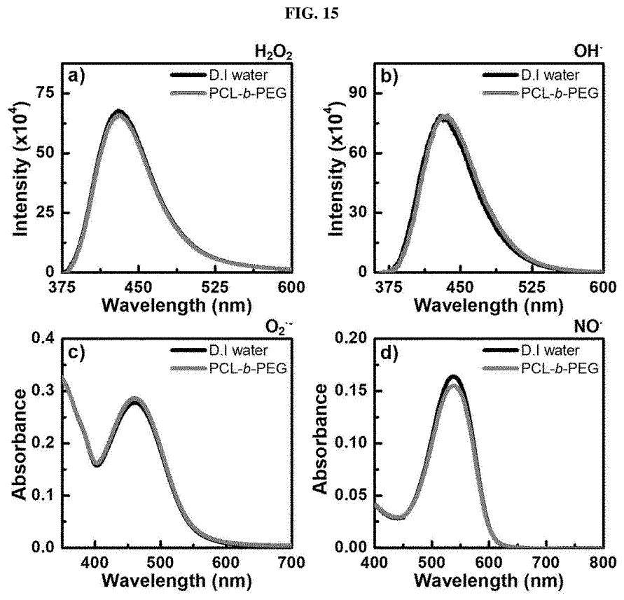

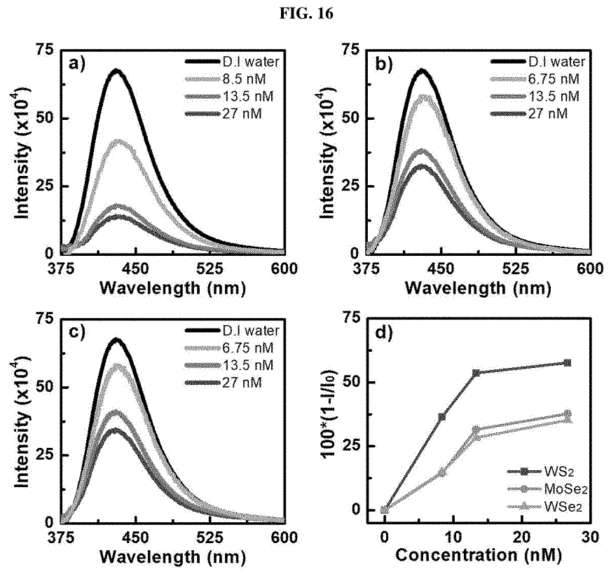

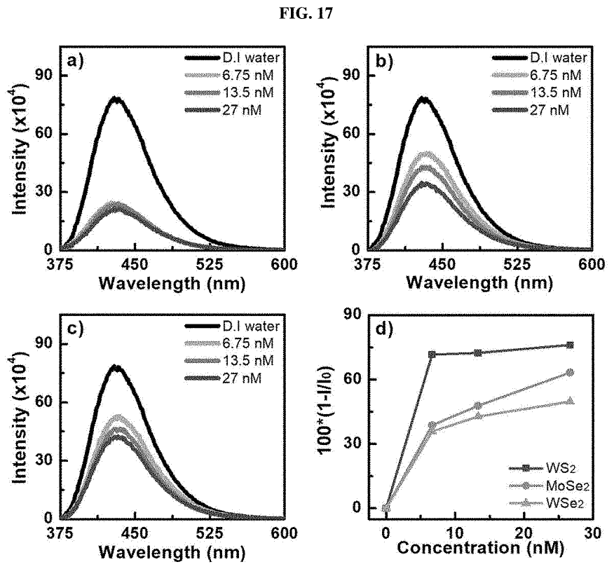

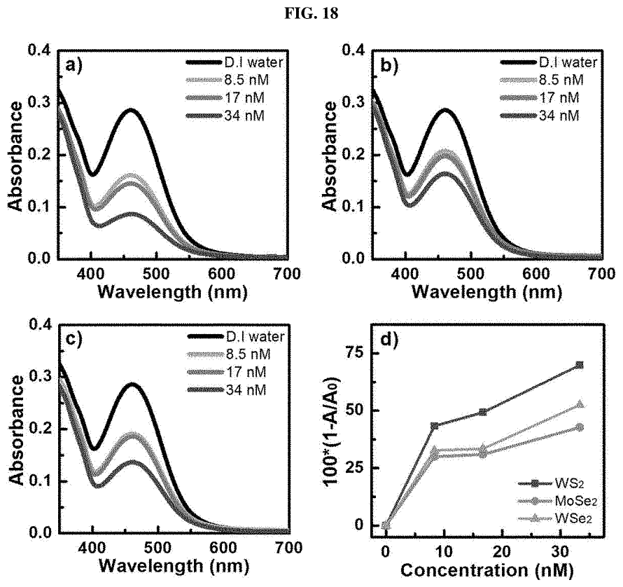

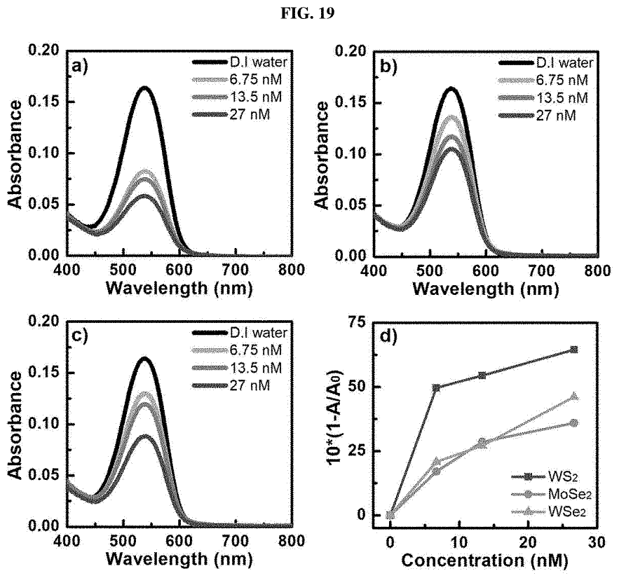

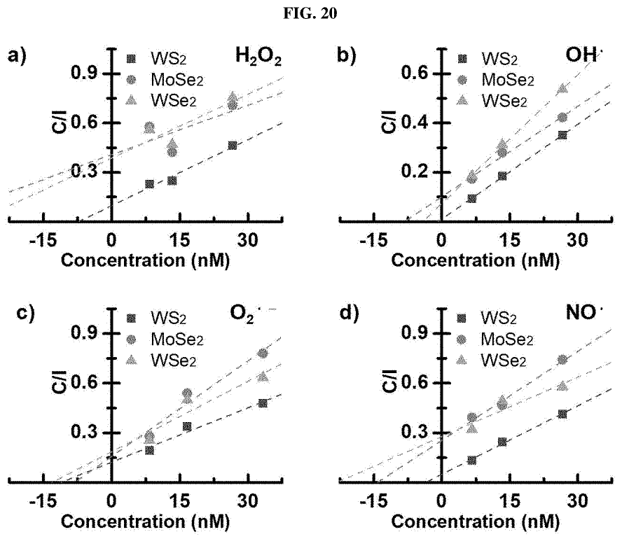

Nitric oxide (NO) can be produced spontaneously from sodium nitroprusside (SNP) at physiological pH. Nitric oxide can be quantitated by Griess reaction after converting all nitrate (NO³⁻) to nitrite (NO²⁻) with nitrate reductase. In the presence of NO in solution, strong absorption at 540 nm was observed after the reaction of the Griess reagent with nitrite converted from NO (FIG. 2f, black line). However, in the presence of the WS₂, MoSe₂ or WSe₂ nanosheet, the absorption was decreased significantly, indicating that NO was effectively scavenged by the TMD nanosheets. The W-based TMD nanosheet showed higher NO scavenging activity than the Mo-based nanosheet. In particular, the WS₂ nanosheet was the most effective in removing NO. In addition, it was confirmed that the activity of scavenging hydroxyl radicals and nitric oxide was maintained as shown in FIGS. 13 and 14. The PCL-b-PEG polymer itself had no scavenging activity for ROS and RNS (FIG. 15). For quantification of the ROS and RNS scavenging activity of the TMD nanosheets, the concentration-dependent ROS and RNS scavenging activity was measured and monitored using equilibrium constants (Kₛ) from the Langmuir isotherm data. As can be seen from FIGS. 16-19, the amount of hydrogen peroxide, hydroxyl radicals, superoxide and nitric oxide was decreased depending on the concentration of the WS₂, MoSe₂ or WSe₂ nanosheet. FIG. 20 shows fitted plots which provide the Kₛ values of each TMD nanosheet for scavenging ROS and RNS (FIG. 2g). As expected, the WS₂ nanosheets had larger Kₛ values for most of the ROS and RNS, which suggests that the WS₂ nanosheets would exert the most effective suppression of aberrant inflammatory response in sepsis.

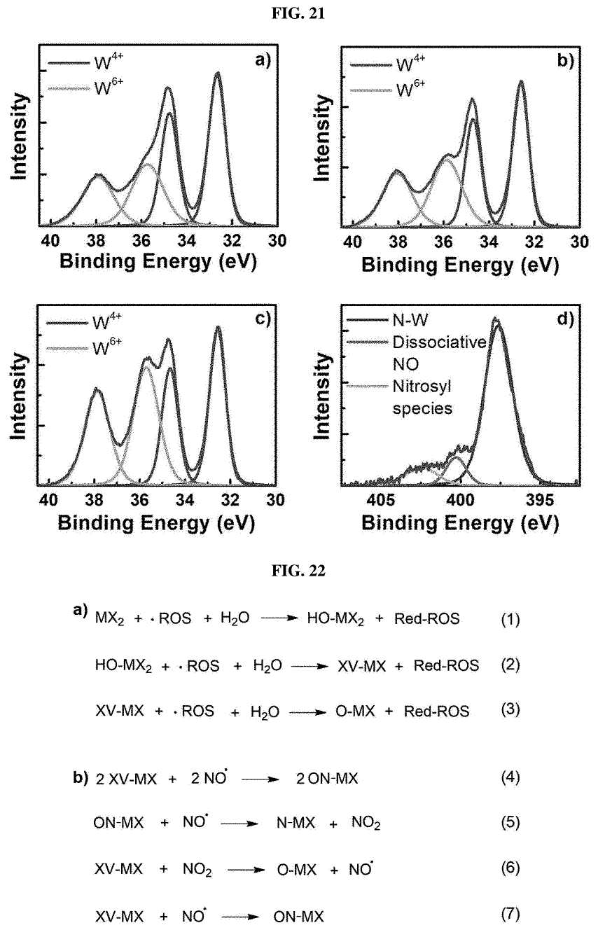

Subsequently, the oxidation state of the WS₂ nanosheets was investigated by XPS analysis and the mechanism responsible for the ROS and RNS scavenging of the TMD nanosheets was investigated. As can be seen from the XPS spectra (FIG. 21), the proportion of W⁶⁺ of the WS₂ nanosheet was increased from 44% to 48% after the H₂O₂ scavenging. After the removal of NO, the proportion of W⁶⁺ in the WS₂ nanosheet was also increased from 44% to 55%. In addition, a peak corresponding to the N—W bond was found clearly at 397.6 eV. After the NO scavenging, two additional peaks corresponding to dissociative NO and nitrosyl species appeared in the Nis XPS spectrum of the WS₂ nanosheet at 400.3 and 402.5 eV, respectively (FIG. 21d).

The ROS and RNS scavenging mechanism of the TMDs could be understood from these results (FIG. 22). The TMDs can be hydroxylated in aqueous solution after ROS scavenging, and the TMDs scavenge ROS in aqueous solution to produce chalcogen-vacant TMDs (XV-MX). The chalcogen-vacant TMDs (XV-MX) further scavenge ROS to produce oxidized TMDs (O-MX). In addition, TMDs react with NO to form nitrosyl-TMD complexes (ON-TMD), followed by dimerization of NO to liberate NO2 as reported in other metal-nitrosyl complexes. Then, the chalcogen-vacant TMDs react with NO2 to form oxidized TMDs (O-MX).

### Example 3. Confirmation of Mitochondrial and Intracellular ROS and RNS Scavenging Activity TMD Nanosheets

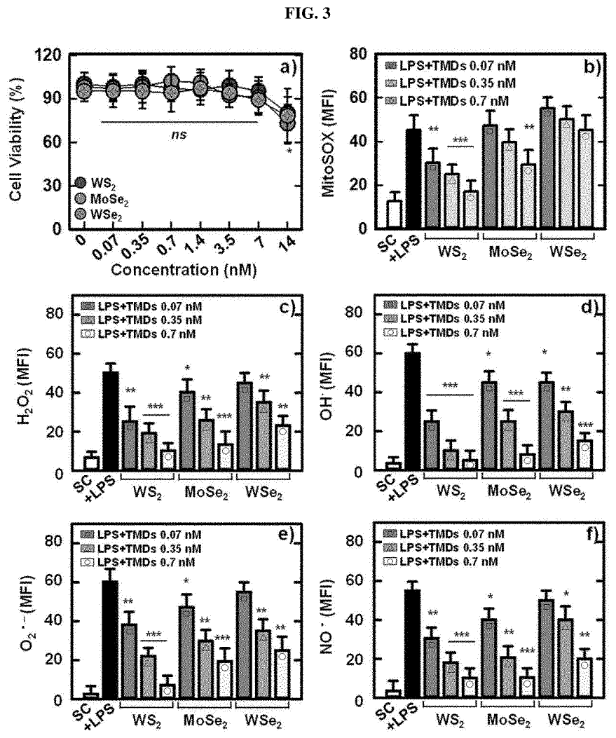

First, the cytotoxicity of WS₂, MoSe₂ and WSe₂ was investigated in macrophages. As can be seen from FIG. 3a, the TMD nanosheets had no or little toxicity.

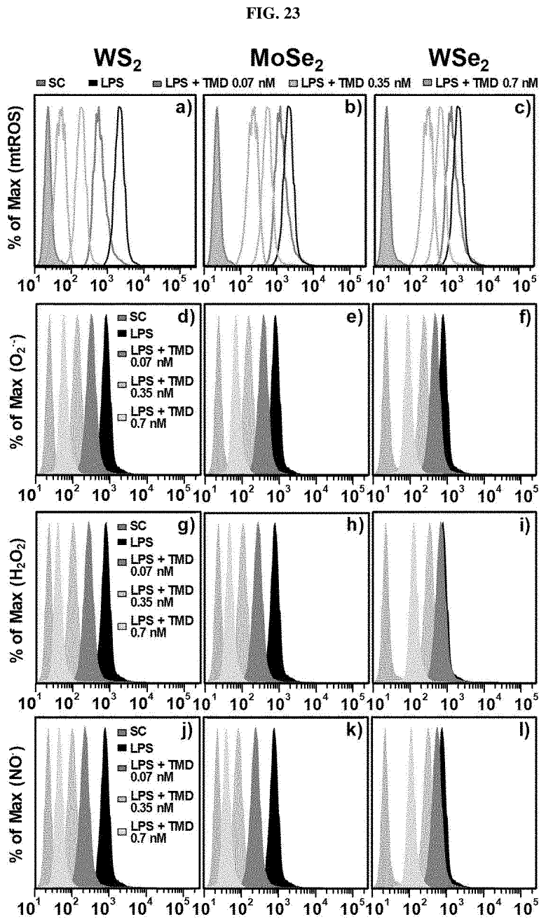

Then, the ROS scavenging activity of the TMD nanosheets in mitochondria was measured using LPS-induced inflammatory cells. As can be seen from FIG. 3b, the mitochondrial ROS level in inflammatory BMDMs (bone marrow-derived macrophages) was increased greatly when they were stimulated with LPS/TLR4 (toll-like receptor 4) (black bar). However, in the presence of the TMD nanosheets, there was little change in the ROS level for the same stimulation. In particular, the WS2 nanosheet showed the best mitochondrial ROS scavenging activity. In addition, the TMDs also suppressed the increase of hydrogen peroxide, hydroxyl radicals, superoxide and nitric oxide in BMDMs stimulated with LPS, indicating that the TMD nanosheets have scavenging activity for intracellular ROS and RNS (FIG. 3c-e, FIG. 23). From these results, it can be seen that the TMD nanosheets can effectively suppress oxidative stress in cells by scavenging both intracellular and mitochondrial ROS and RNS.

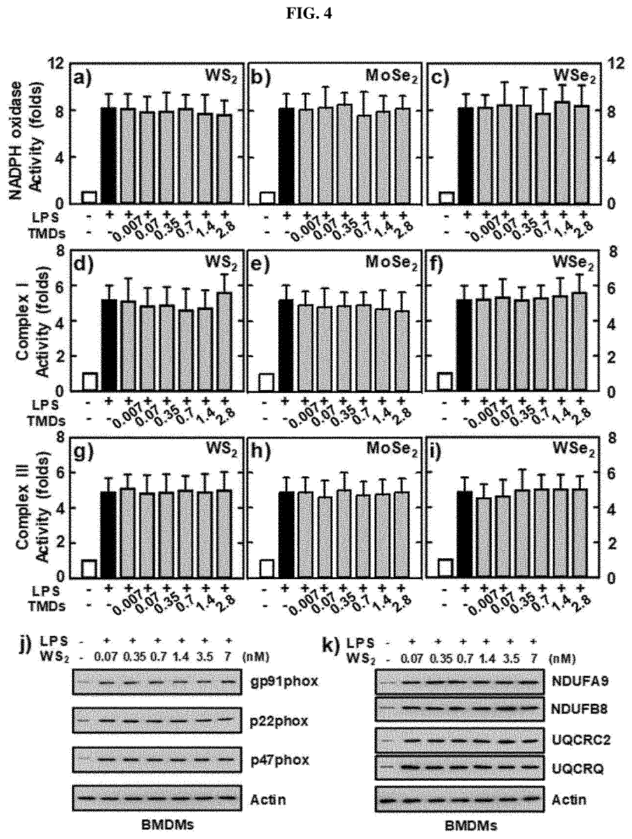

Then, it was investigated by measuring the activity of enzymes involved in the production of ROS in macrophages stimulated with LPS whether the TMDs inhibit the activity of the enzymes. Specifically, the activity of NADPH oxidase (NOX) involved in the formation of intracellular ROS and OXPHOS (mitochondrial oxidative phosphorylation) complexes I & III involved in the formation of mitochondrial ROS was investigated. As can be seen from FIG. 4a-i, the TMD nanosheets did not inhibit the activity of NOX and OXPHOS complexes I & III in macrophages, and the enzymes showed consistent activities in the presence of the TMDs. In addition, as can be seen from FIGS. 4j and 4k, the TMD nanosheets did not affect the expression level of NOX subunits (gp91phox, p22phox and p47phox) and OXPHOS subunits (NDUFA9 and NDUFB8 for complex I; UQCRC2 and UQCRQ for complex III).

From these result, it can be seen that, although the TMD nanosheets effectively remove ROS and RNS in cells and mitochondria, they have no effect on the activity and expression of ROS-producing enzymes in macrophages stimulated with LPS.

### Example 4. Confirmation of Inflammatory Cytokine Inhibition Activity of TMD Nanosheets

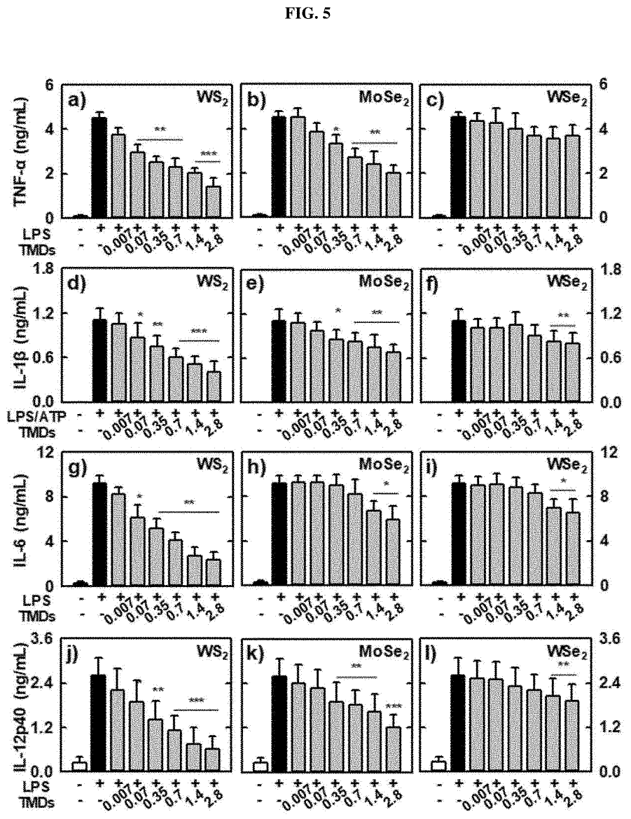

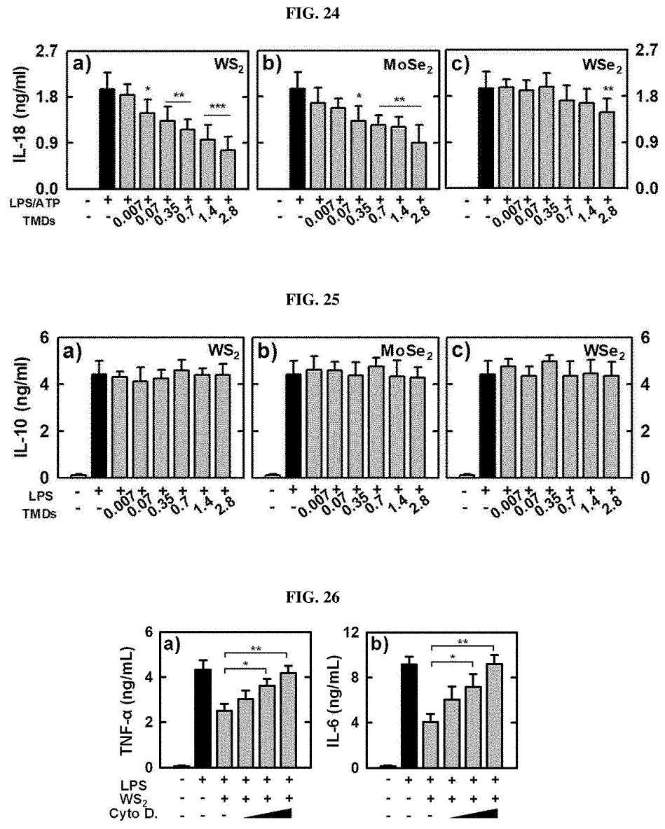

The increased level of ROS and RNS activates immune cells in sepsis, TLR4 activation of immune cells leads to organ damage. Therefore, it was investigated whether the TMD nanosheets affect the inflammatory response regulated by ROS. It was confirmed that the TMD nanosheets functionalized with PCL-b-PEG effectively inhibits the excessive secretion of acute inflammatory cytokines including TNF-α, IL-1β and IL-18 in a concentration-dependent manner (FIG. 5a-f and FIG. 24). In addition, the TMD nanosheets significantly inhibited chronic inflammatory cytokines such as IL-6 and IL-12p40 (FIG. 5g-l). In particular, the WS2 nanosheet showed significant inhibition activity for both acute and chronic inflammatory cytokines even at a very low concentration. However, the three TMD nanosheets had no effect on the secretion of the anti-inflammatory cytokine (IL-10) (FIG. 25). From these results, it can be seen that the TMD nanosheets can not only scavenge intracellular and mitochondrial ROS and RNS but also effectively inhibit the excessive secretion of pro-inflammatory cytokines.

In order to further understand the intracellular regulatory mechanism of the TMD nanosheets for scavenging of ROS and RNS and inhibition of inflammation in macrophages, Raw264.7 cells were pre-incubated with cytochalasin D. Cytochalasin D can completely block the introduction of the TMD nanosheets into cells by inhibiting the polymerization of actin. As can be seen from FIG. 26, the secretion of inflammatory cytokines (TNF-α and IL-6) was increased in proportion to the concentration of cytochalasin D despite the addition of the WS₂ nanosheet. In contrast, it was confirmed from ICP-AES (inductively coupled plasma-atomic emission spectroscopy) analysis that the TMD nanosheets were effectively introduced into cells in proportion to concentration when the cells were not pre-treated with cytochalasin D (FIG. 16c). From these results, it can be seen that the introduction of the TMD nanosheets is essential in scavenging ROS and RNS and inhibiting inflammation in macrophages.

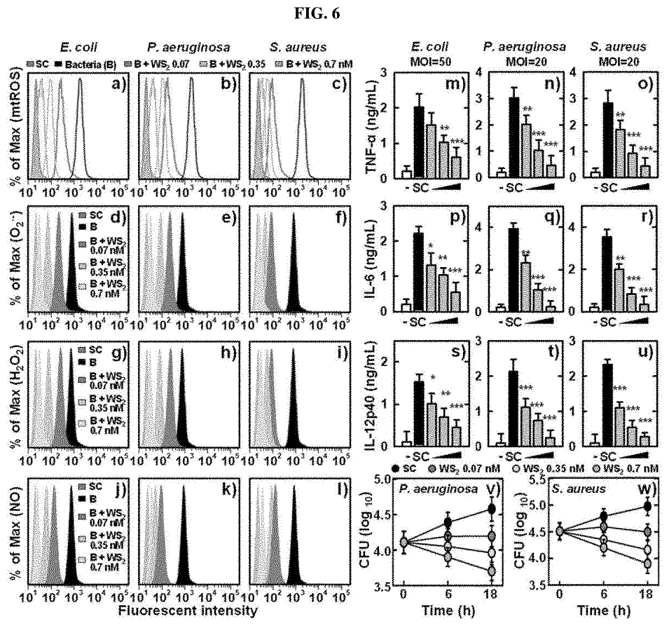

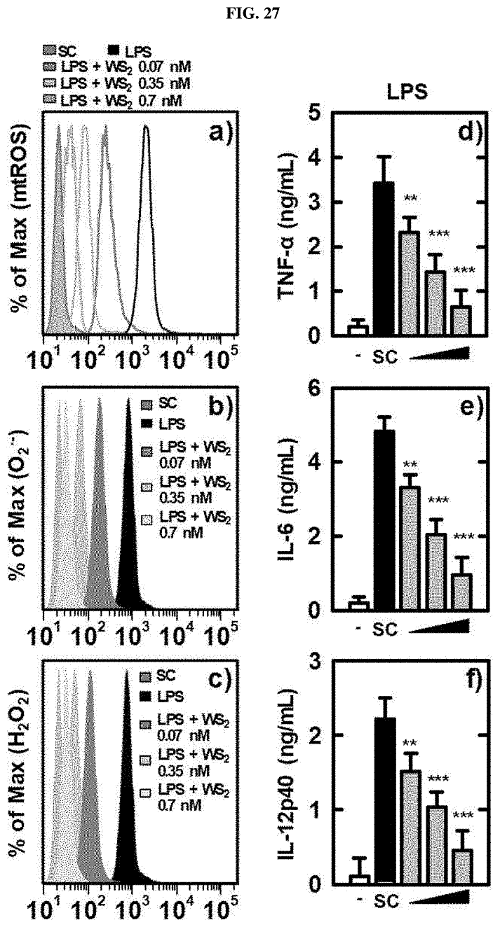

Then, the WS₂ nanosheet showing the best activity among the three TMD nanosheets was applied to human monocytes and its activity of scavenging ROS and RNS and inhibiting inflammatory cytokines was investigated. Specifically, monocytes were infected with common bacteria such as E. coli, P. aeruginosa, S. aureus, etc. or stimulated with LPS. As can be seen from FIG. 27, the WS₂ nanosheet effectively removed intracellular and mitochondrial ROS and RNS and inhibited excessive expression of pro-inflammatory cytokines (TNF-α, IL-6 and IL-12p40) in the human monocytes stimulated with LPS. Additionally, the WS₂ nanosheet significantly decreased the concentration of ROS and RNS in the mitochondria and cytoplasm of human monocytes infected with sepsis-causing bacteria (FIG. 6a-1). In addition, the WS₂ nanosheet significantly decreased the secretion of inflammatory cytokines even at very low concentrations (FIG. 6m-u). Interestingly, the number of bacteria in the monocytes was decreased as the concentration of the WS₂ nanosheet was increased (FIGS. 6v and w).

From these results, it can be seen that, since the WS2 nanosheet decreases the excessive secretion of inflammatory cytokines and scavenges intracellular and mitochondrial ROS and RNS in sepsis, it can be used as an effective antioxidant for treating sepsis by inhibiting inflammatory response.

### Example 5. Confirmation of Therapeutic Effect of TMD Nanosheets for Sepsis In Vivo

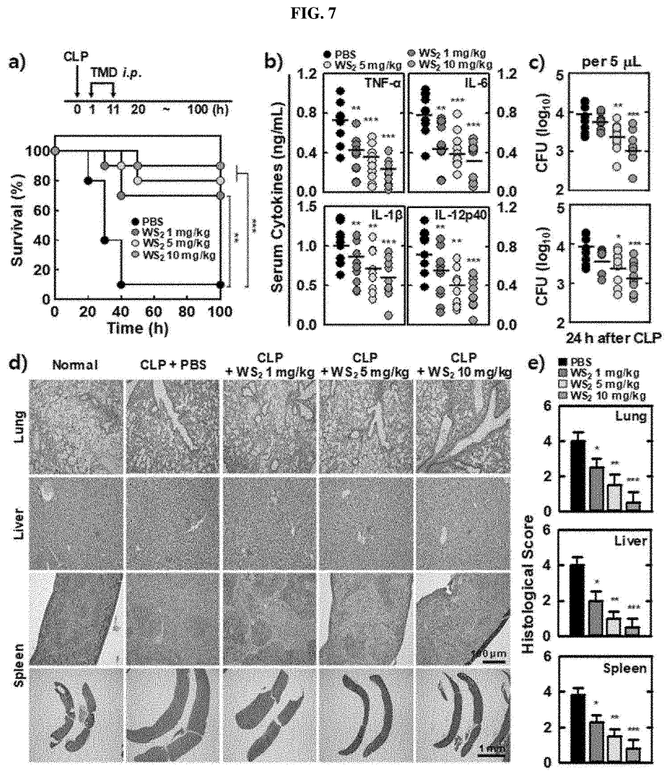

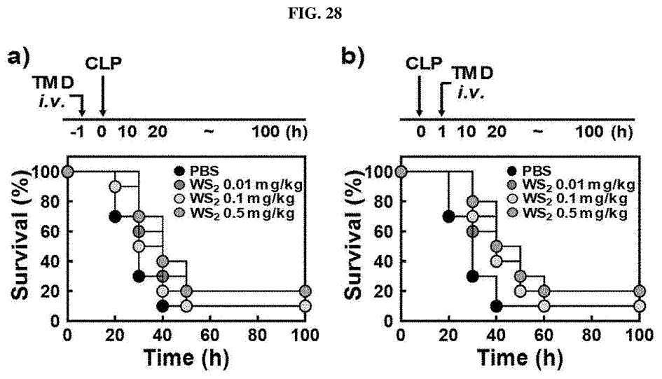

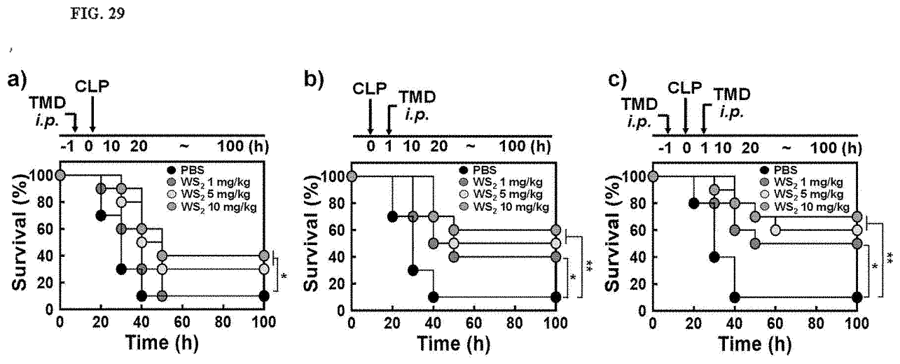

Then, the therapeutic effect of the WS₂ nanosheet for sepsis was investigated for an animal model of severe sepsis. A CLP (cecal ligation and puncture)-induced bacteremic mouse model, which is the most similar to human microbial infection, was used, and the administration dose, administration route and administration period of the WS₂ nanosheet were varied (FIGS. 28-29). The survival rate of septic mice was increased depending on the administration dose of the WS₂ nanosheet when it was injected intraperitoneally twice at 1 hour and 11 hours. The survival rate reached 90% at the WS₂ nanosheet dose of 10 mg/kg (FIG. 7a). The survival rate was higher although the administration dose of the WS₂ nanosheet was much lower than that of other nanomaterials known as antioxidants in previous researches (administration dose of the WS₂ nanosheet: 2.39 nmol, survival rate of mice when other antioxidants were administered: 60%). In addition, the level of pro-inflammatory cytokines such as TNF-α, IL-6, IL-1β and IL-12p40 was decreased significantly in the serum of the septic mice treated with the WS₂ nanosheet (FIG. 7b).

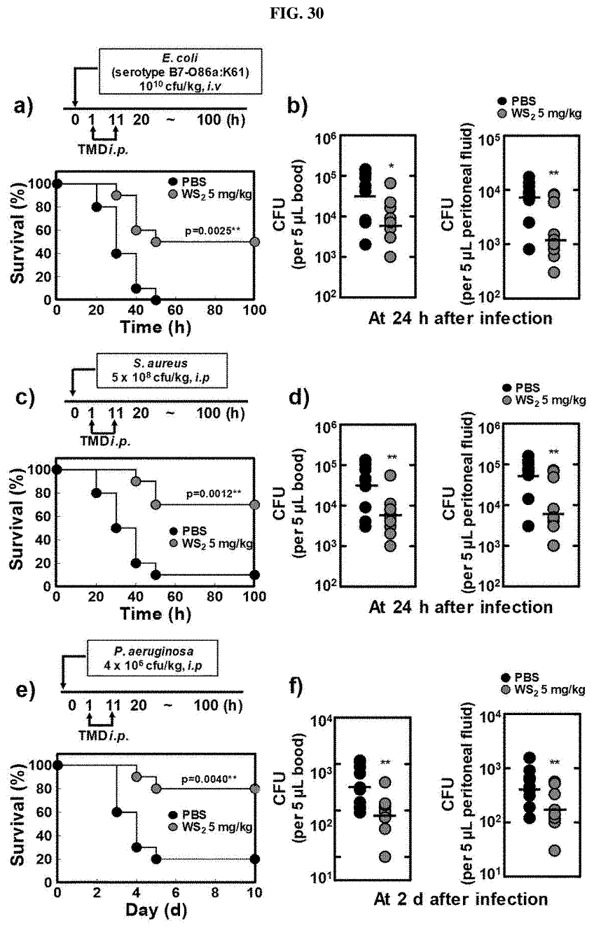

The therapeutic effect of the WS₂ nanosheet antioxidant was similarly manifested in the animal model of severe sepsis. Additionally, the number of bacteria in the septic mice was counted after the treatment with the WS₂ nanosheet because there is a positive correlation between CLP-induced lethality and the number of bacterial colonies in peripheral blood and peritoneal fluid. As can be seen from FIG. 7c, the number of bacterial colonies was decreased significantly in both the blood (top) and peritoneal fluid (bottom) of the CLP-induced bacteremic mouse model treated with the WS₂ nanosheet. From these results, it can be seen that the administration of the WS₂ nanosheet to the sepsis model infected with E. coli, S. aureus or P. aeruginosa decreased the number of bacterial colonies and increased survival rate (FIG. 30).

Furthermore, histological analysis was performed on the liver, lung and spleen tissues. Specifically, the thickness of the alveolar wall and the necrosis of liver and spleen cells was compared with normal tissues by staining the tissues of the CLP-induced bacteremic mice with H&E (hematoxylin & eosin) (FIG. 7d, CLP+PBS). The septic mice treated with the WS₂ nanosheet (CLP+WS₂) showed significantly decreased histological pathologies in proportion to the concentration. The histological pathologies based on the number and distribution of inflammatory cells in tissues and the damage and repair of the bronchial epithelium showed that the severity of sepsis is much lower in the WS₂-treated CLP mice.

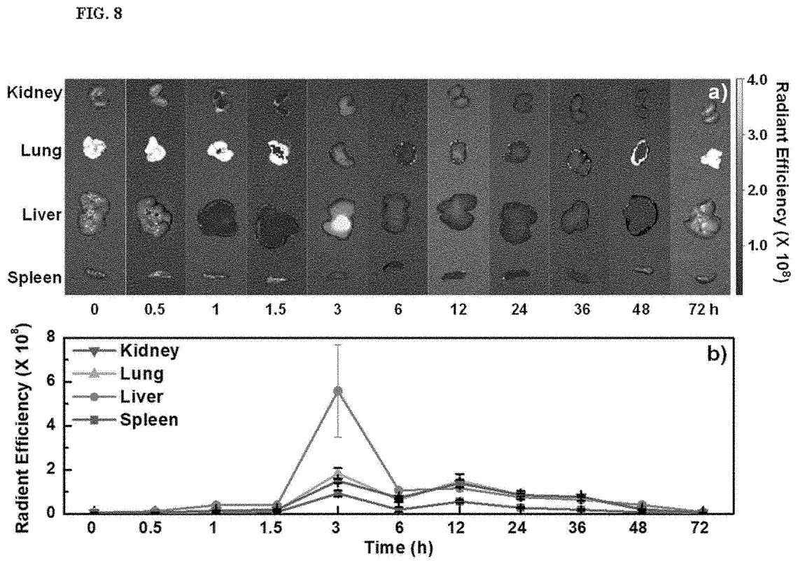

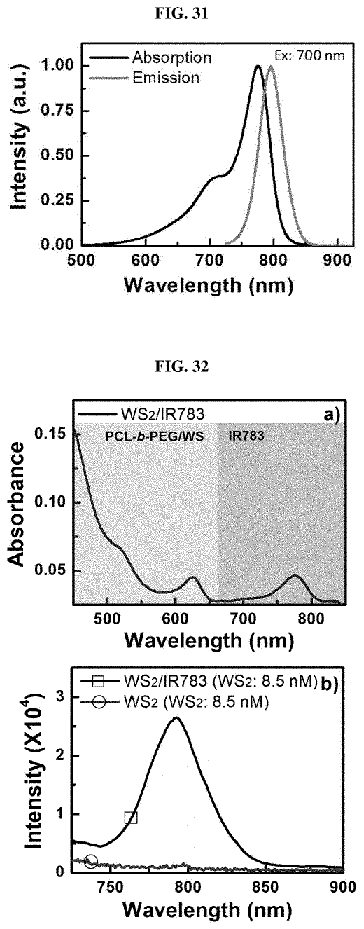

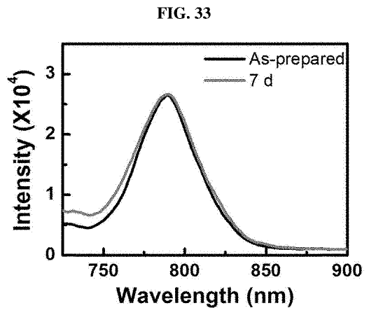

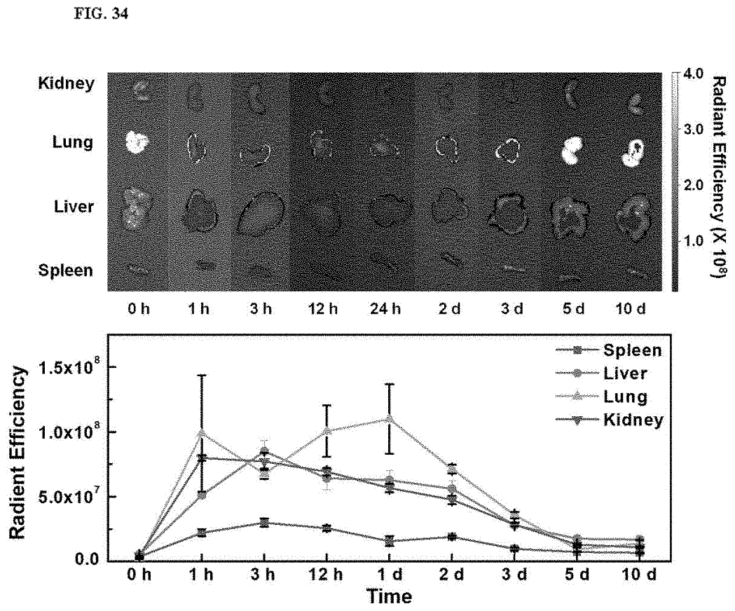

Finally, the biodistribution and pharmacokinetics of the WS₂ nanosheet were investigated using a CT (IVIS spectrum-chromatography) system. The WS₂ nanosheet was functionalized with a fluorescent dye (IR783, FIG. 31) and then intravenously injected to mice. The WS₂ nanosheet including the fluorescent dye showed strong and stable fluorescence at 800 nm over time (FIGS. 32-33). As can be seen from the fluorescence images of several organs (FIG. 8), the WS₂ nanosheet was first accumulated in the liver within 1 hour, followed by distribution in other organs, such as spleen, lung and kidney, within 3 hours. It was completely discharged from the organs of the mice in 3 days after the injection. In addition, when the WS₂ nanosheet was injected intraperitoneally, it was distributed in different organs from 1 hour and remained in the organs of the mice for 10 days (FIG. 34). It is thought that the WS₂ nanosheet is discharged from the organs of the mice faster than in the previous studies because it has a smaller and thinner nanostructure. This result suggests that the therapeutic WS₂ nanosheet has high potential for treatment of sepsis in clinical trials as well as biocompatibility.

While the specific exemplary embodiments of the present disclosure have been described in detail, it will be obvious to those having ordinary knowledge in the art that they are merely preferred exemplary embodiments and the scope of the present disclosure is not limited by them. Accordingly, it is to be appreciated that the substantial scope of the present disclosure is defined by the appended claims and their equivalents.

1. A pharmaceutical composition for preventing or treating sepsis or septic shock, comprising a 2D transition metal dichalcogenide (TMD) functionalized with an amphiphilic block polymer compound comprising a hydrophilic block and a hydrophobic block as an active ingredient.

2. The pharmaceutical composition of claim 1, wherein the hydrophilic block is polyethylene glycol (PEG).

3. The pharmaceutical composition of claim 1, wherein the hydrophobic block is poly(ε-caprolactone) (PCL).

4. The pharmaceutical composition of claim 1, wherein the amphiphilic block polymer compound comprises PEG and PCL and has a structure of Chemical Formula 1: 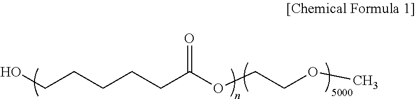
wherein n is an integer from 400 to 3000.

5. The pharmaceutical composition of claim 1, wherein the TMD is one or more selected from the group consisting of tungsten disulfide (WS₂), molybdenum diselenide (MoSe₂), and tungsten diselenide (WSe₂).

6. The pharmaceutical composition of claim 1, wherein the functionalized 2D TMD has a thickness of 1-50 nm.

7. The pharmaceutical composition of claim 1, wherein the functionalized 2D TMD has a lateral length of 1-500 nm.

8. The pharmaceutical composition of claim 1, wherein the functionalized 2D TMD has the activity of scavenging intracellular and/or mitochondrial ROS and/or RNS.

9. The pharmaceutical composition of claim 1, wherein the functionalized 2D TMD inhibits the secretion of an inflammatory cytokine.

10. The pharmaceutical composition of claim 9, wherein the inflammatory cytokine is TNF-α, IL-1β, IL-6, IL-18 or IL-12p40.

11. A method for preventing or treating sepsis or septic shock, comprising a step of administering a 2D transition metal dichalcogenide (TMD) functionalized with an amphiphilic block polymer compound comprising a hydrophilic block and a hydrophobic block to a subject.

12. A use of a 2D transition metal dichalcogenide (TMD) for preparation of a medication for preventing or treating sepsis or septic shock, wherein the 2D TMD is functionalized with an amphiphilic block polymer compound comprising a hydrophilic block and a hydrophobic block.

13. The method of claim 11, wherein the hydrophilic block is polyethylene glycol (PEG).

14. The method of claim 11, wherein the hydrophobic block is poly(ε-caprolactone) (PCL).

15. The method of claim 11, wherein the amphiphilic block polymer compound comprises PEG and PCL and has a structure of Chemical Formula 1: 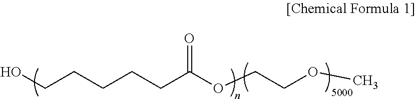
wherein n is an integer from 400 to 3000.

16. The method of claim 11, wherein the TMD is one or more selected from the group consisting of tungsten disulfide (WS₂), molybdenum diselenide (MoSe₂), and tungsten diselenide (WSe₂).

17. The method of claim 11, wherein the functionalized 2D TMD has a thickness of 1-50 nm.

18. The method of claim 11, wherein the functionalized 2D TMD has a lateral length of 1-500 nm.

19. The method of claim 11, wherein the functionalized 2D TMD has the activity of scavenging intracellular and/or mitochondrial ROS and/or RNS.

20. The method of claim 11, wherein the functionalized 2D TMD inhibits the secretion of an inflammatory cytokine.
